(19) United States
(12) Patent Application Publication
(10) Pub. No.: US 2009/0259245 A1
(43) Pub. Date: Oct. 15, 2009

(54) VIBRATING KNIFE AND EXCISION APPARATUS

(75) Inventors: Tadashi Hayashi, Kanagawa (JP); Yasushi Miura, Kanagawa (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Tokyo (JP)

(21) Appl. No.: 12/491,348

(22) Filed: Jun. 25, 2009

Related U.S. Application Data
(62) Division of application No. 10/810,827, filed on Mar. 29, 2004.

(30) Foreign Application Priority Data
Apr. 7, 2003 (JP) 2003-103496

(51) Int. Cl. A61B 17/32

(52) U.S. Cl. 606/169

(57) ABSTRACT

Demands have arisen for a vibrating knife such as a biometrical ultrasonic knife which can easily obtain sharp cut surfaces with little possibility of contamination and the like. In order to meet the demands, the excision portion of a vibrating knife such as a biometrical ultrasonic knife which is brought into contact with an excision target such as living body tissue and vibrated in a direction at an angle to the traveling direction in excision so as to excise the target is coated such that the surface of the portion located on the forward side in the traveling direction in excision becomes hydrophobic, and the surface of the portion located on the backward side in the traveling direction in excision becomes hydrophilic.

### FIELD OF THE INVENTION

The present invention relates to a vibrating knife and excision apparatus and, more particularly, to a vibrating knife, a biomedical ultrasonic knife, and an excision apparatus which cut off minute tissue, cells, and the like from a biomaterial which is used for biotechnology, medical tests, and the like.

### BACKGROUND OF THE INVENTION

As a biomedical excision apparatus designed for active control, an ultrasonic knife having an arrangement like that shown in FIG. 8 is known (see Japanese Patent Laid-Open No. 64-70036). In this arrangement, a displacement enlarging horn 101 is attached to the leading end of bolted Langevin transducer 100. The Langevin transducer 100 generates longitudinal vibrations. The vibrations are transmitted to the leading end to perform excision. According to this arrangement, the vibration amplitude of the transducer 100 is magnified about 300 times at the leading end. This makes it possible to cut tissue without attaching any sharp blade.

As an apparatus having another arrangement, a cutting apparatus designed to burn out tissue by focusing a laser beam or microwaves onto a region to be cut has been proposed. According to this arrangement, since noncontact cutting can be done, there is little possibility of contamination due to contact.

In the above biometrical ultrasonic knife of the contact type, however, the excision direction tends to be unstable because of the use of simple longitudinal vibrations. This tendency is conspicuous with respect to biomaterials, in particular, because they are soft, having no rigidity unlike a general excision target material such as a metal. In addition, owing to the contact type, part of tissue adheres and sticks to the knife surface after excision. Because of these two reasons, it is difficult to cut a target region with a sharp cut surface. Furthermore, a tissue portion adhering to the knife may become a source of contamination.

According to the above method using laser beams or microwaves, the apparatus has larger scale and higher cost than a contact type ultrasonic knife. In addition, setting and usage are difficult, and hence sophisticated knowledge and skill are required. Furthermore, since this method is a method of burning out a target region by converting optical energy into thermal energy, when such energy is applied to a material that tends to denature like proteins, the properties of the material change. This makes it impossible to perform an accurate test after the preparation of a sample.

Demands have therefore arisen for a vibrating knife such as a biometrical ultrasonic knife which can easily obtain sharp cut surfaces with little possibility of contamination or the like, and an excision apparatus using such a knife.

### SUMMARY OF THE INVENTION

According to the first aspect of the present invention, a vibrating knife such as a biomedical ultrasonic knife includes an excision member which is brought into contact with a target including living body tissue like cells and vibrated in a direction at an angle (typically, a right angle direction) to the traveling direction in excision so as to excise the target, and the surface of a portion of the excision member which is located on the forward side in the traveling direction in excision becomes one of a hydrophobic surface and a hydrophilic surface, while the surface of a portion of the excision member which is located on the backward side in the traveling direction in excision becomes the other of the hydrophobic surface and the hydrophilic surface.

A knife having such an arrangement is generally vibrated by ultrasonic waves. However, as long as an excision function is fulfilled, the knife can be vibrated in a vibration mode other than ultrasonic vibration in a strict sense, which is by way of example, vibration near ultrasonic vibration. An excision target is typically living body tissue which exhibits hydrophobicity, and the knife is used with its hydrophobic surface serving as a leading end face. If an excision target is hydrophilic, the knife is used with its hydrophilic surface serving as a leading end face. Consider the sectional shape of the knife. When excision is to be performed by vibrations such as ultrasonic waves, the portion used for excision need not be very sharp. For example, it suffices if this portion has a streamline shape. The overall sectional shape may be determined in consideration of, for example, whether or not the two end portions are to be used for excision, whether or not the shape is well balanced in terms of desired vibration, whether the knife can be easily manufactured, whether the shape has enough strength, and whether the knife can be easily handled.

The knife according to each embodiment of the present invention can therefore excise an excision target by using a surface having the same property as that of the target as a leading end face and using the trailing end face as a surface having a property different from that of the target depending on whether the target is hydrophobic or hydrophilic. This makes it possible to make the excision target adhere well to the surface of the leading end portion of the knife, thus reducing the instability of the excision direction. In addition, this can make the target after excision separate well from the knife to prevent the target from adhering to the knife again. Therefore, a sharp cut surface can be easily obtained, and there is little possibility of contamination. The hydrophobic surface and hydrophilic surface can be formed by making a hydrophobic film and hydrophilic film adhere to the main body of the excision portion (see FIG. 1).

According to the second aspect of the present invention, a vibrating knife such as a biomedical ultrasonic knife includes an excision member which is brought into contact with a target and vibrated in a direction at an angle to the traveling direction in excision so as to excise the target, a material whose property changes to hydrophilicity or hydrophobicity depending on the temperature (e.g., a temperature-responsive polymer) is provided on the surface of the excision member, and a temperature control unit such as a heater element is provided near the surface of a portion of the excision member which is located on the forward side in the traveling direction in excision or the surface of a portion of the excision member which is located on the backward side in the traveling direction in excision. The function of the knife according to the second aspect is basically the same as that of the knife according to the first aspect.

According to the third aspect of the present invention, a vibrating knife such as a biomedical ultrasonic knife includes an excision member which is brought into contact with a target and vibrated in a direction at an angle to the traveling direction in excision so as to excise the target, a material whose property changes to hydrophilicity or hydrophobicity depending on the temperature is provided on the surface of the excision member. The function of the knife according to the third aspect is basically the same as that of the knife according to the first aspect. In this case, the excision member can be formed such that the vibration amplitude on the surface of a portion located on the forward side in the traveling direction in excision becomes relatively large, and the surface exhibits hydrophobicity (see FIG. 6).

In the knives according to the first to third aspects, each excision member can be formed in the shape of a horn or the like which generates necessary enlarged vibrations (see FIGS. 6 and 7). In addition, as in the embodiments to be described later, each knife can be formed as a biomedical ultrasonic knife having a hydrophobic surface located on the forward side in the traveling direction in excision, and a hydrophilic surface located on the backward side in the traveling direction in excision.

According to the fourth aspect of the present invention, there is disclosed a vibrating excision apparatus including the vibrating knife described above, a knife driving portion (e.g., Langevin transducer including a piezoelectric element) which vibrates the vibrating knife by generating vibrations, and a driving control portion which controls the knife driving portion and vibration of the knife. This excision apparatus can be directly operated by an operator, and may be mounted on a manipulating apparatus such as a manipulator or robot hand which manipulates a knife apparatus.

The excision member can be formed in the shape of a horn which generates necessary enlarged vibrations. However, a vibration enlarging member which is connected to the excision member and generates necessary enlarged vibrations may be provided. Furthermore, the knife driving portion and driving control portion can be formed to generate elliptic vibration whose ellipsoid coincides with the traveling direction in excision at the leading end of the excision member (see FIG. 7).

Other features and advantages of the present invention will be apparent from the following description taken in conjunction with the accompanying drawings, in which like reference characters designate the same or similar parts throughout the figures thereof.

### BRIEF DESCRIPTION OF THE DRAWINGS

FIG. 1 is a sectional view showing how living body tissue is incised by using an ultrasonic knife according to the first embodiment of the present invention;

FIG. 2 is a sectional view showing how living body tissue is incised by using an ultrasonic knife;

FIG. 3 is a schematic view showing a cell and adhesive proteins on the surface of the cell;

FIG. 4 is a sectional view showing how living body tissue is incised by using an ultrasonic knife according to the second embodiment of the present invention;

FIG. 5 is a sectional view showing how living body tissue is incised by using an ultrasonic knife according to the third embodiment of the present invention;

FIG. 6 is a front view showing a form of an ultrasonic knife according to the third embodiment of the present invention;

FIG. 7 is a view showing the arrangement of the fourth embodiment of the present invention and the vibration mode of the leading end of a knife; and

FIG. 8 is a view showing an ultrasonic excision apparatus.

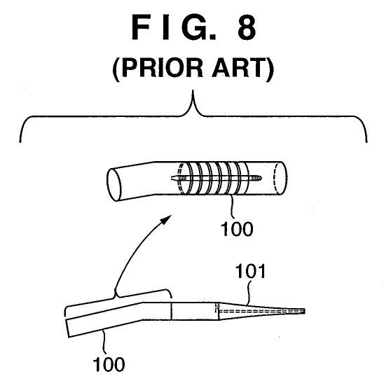

### DETAILED DESCRIPTION OF THE PREFERRED EMBODIMENTS

The embodiments of the present invention will be described in detail below with reference to the accompanying drawings.

### First Embodiment

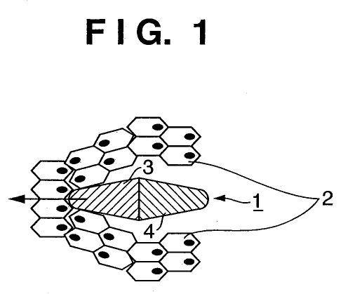

FIG. 1 is a sectional view showing how living body tissue is incised by using an ultrasonic knife according to the first embodiment of the present invention. Referring to FIG. 1, reference numeral 2 denotes a cell; and 3 and 4, portions of a single-piece knife 1 which have different properties. The knife 1 incises living body tissue in the direction indicated by the arrow. In this incision, the knife 1 tears off the living body tissue upon being provided with ultrasonic vibrations in a direction perpendicular to the drawing surface. The surface of one portion 3 of the knife 1 whose main body is made of glass or the like is covered with a hydrophobic coating, whereas the surface of the other portion 4 is covered with a hydrophilic coating.

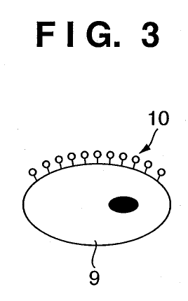

As shown in FIG. 3, in general, the surface of a cell 9 is covered with adhesive proteins 10, and exhibits hydrophobicity. In the first embodiment, almost the half portion of the knife 1 which is located on the forward side in the traveling direction in incision is the hydrophobic coating portion 3, and hence the cells 2 adhere well to the knife 1. This reduces the instability of the traveling direction of the knife 1 in incision due to the flexibility of living body tissue. More specifically, as shown in FIG. 1, the cells 2 stick to the hydrophobic coating portion 3. In contrast, almost the half portion of the knife 1 which is located on the backward side in the traveling direction of the knife 1 in incision is the hydrophilic coating portion 4. This prevents the cells 2 and like from re-adhering to the knife 1 due to the adhesive proteins 10. In addition, since living body tissue repels the hydrophilic coating portion 4 of the knife 1, the cells 2 of the incised living body tissue separate well from the knife 1.

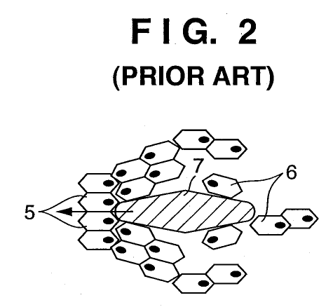

In contrast, FIG. 2 shows an incision in living body tissue with a general ultrasonic knife 7 (FIG. 2 shows the same sectional shape as that of the knife 1 in FIG. 1 for the sake of comparison). In this case, the entire surface of the knife 7 is made of a material exhibiting hydrophobicity. Since the surface of the ultrasonic knife 7 is hydrophobic, cells 5 near the forward side in the traveling direction in incision adhere well to the knife 7. This reduces the instability of the traveling direction. However, cells 6 and adhesive proteins 10 temporarily separate from the knife 7 and then adhere to the surface of knife (this behavior will be referred to as “re-adhesion” hereinafter) which is located on the backward side in the traveling direction in incision. These accretions may become contamination and invalidate test materials as well as degrading the incision efficiency. In addition, since cells and the like do not separate well from the knife, living body tissue cannot be incised sharply with the knife, resulting in delaying the operation. In contrast to this, if the surface of the knife 7 is hydrophilic, there is no affinity between the forward side (leading end) of the knife in the traveling direction in incision and living body tissue. Since, in addition, the living body tissue is soft, the traveling direction in incision becomes unstable. This makes it impossible to sharply incise the tissue.

As described above, the first embodiment can simultaneously obtain the two effects, that is, ensuring good affinity between living body tissue and the knife on the forward side (leading end) in the traveling direction in incision and making them adhere well to each other, and making the living body tissue and the knife separate well from each other on the backward side (trailing end) in the traveling direction in incision and preventing re-adhesion.

Coating methods to be used include, for example, a dipping method, spray coating method, screen printing method, and vacuum deposition method. In this embodiment, the surface of a knife must be selectively coated, and hence a promising method is the method of making a coating agent containing a binder adhere to the surface of a knife by electrical charging and then fixing the agent by heating, as in electrophotography. Alternatively, if a coating agent can be dissolved in a liquid medium or can be solated, selective coating can be easily done by an ink-jet method. In addition, as in a semiconductor lithography process, a coating material may be formed on the surface of a knife by deposition or the like, a mask member is then formed on the resultant arrangement, and the mask material is exposed and selectively etched to form a pattern.

Another method is the method of forming coatings on the hydrophilic side and hydrophobic side of a knife in advance by an inexpensive method such as coating or dipping and joining the coating with an adhesive or the like.

### Second Embodiment

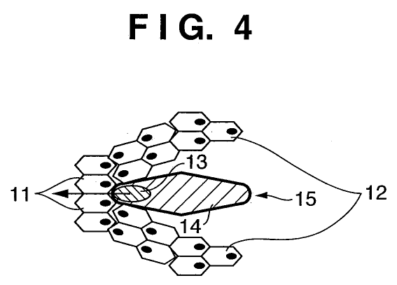

FIG. 4 is a sectional view showing how living body tissue is incised by using an ultrasonic knife according to the second embodiment. Referring to FIG. 4, reference numerals 11 and 12 denote cells; 13, a heater which extends by a proper length in a direction perpendicular to the drawing surface and is provided on the tip portion of a knife 14; and 15, a coating film made of a temperature-responsive polymer such as polyisopropylacrylamide. The properties of polyisopropylacrylamide change at a critical temperature of about 32° C. More specifically, this polymer exhibits hydrophobicity at the critical temperature or higher, and hydrophilicity at a temperature lower than the critical temperature.

Incising with the knife 14 having the above arrangement according to the second embodiment will be described. Living body tissue is generally stored at a low temperature (the critical temperature or lower) to prevent decomposition. In this case, an apparatus used for an incision is also kept at a low temperature, and the knife 14 is kept at the temperature as well. When an incision is to be performed, the heater 13 is energized to control the temperature of the contact surface (tip) between living body tissue and the knife 14 to the critical temperature or higher. At this time, since the temperature at which a test target such as a protein deteriorates is higher than the critical temperature of polyisopropylacrylamide by 20° C. or more, no problem arises if the temperature of the contact surface is held within 20° C. from the critical temperature. With this control, since the contact surface between the knife 14 and the living body tissue as an incision target exhibits hydrophobicity, the cells 11 near the forward side of the knife 14 in the traveling direction in incision adhere well to the knife 14. In addition, since the temperature around the knife 14 is kept low, if temperature control is performed to make the temperature of the rear portion of the knife 14 in the traveling direction in incision have a temperature equal to or lower than the critical temperature, the cells 12 and adhesive proteins separate well from the knife. This can also prevent re-adhesion. In order to enhance this effect, a heat insulating member may be attached between the heater 13 and the rear portion of the knife 14 in the traveling direction in incision so as to prevent the heat of the heater 13 from being transmitted to the rear portion of the knife 14 in the traveling direction in incision.

If, therefore, a coating film having a critical temperature higher than the storage temperature for an incision target and lower than the temperature at which the incision target deteriorates is formed on the surface of the knife 14, and temperature control on the heater 13 is performed by a combination of, for example, a temperature sensor placed near the heater 13 and a temperature controller which supplies power to the heater 13 in accordance with the temperature detected by the temperature sensor, the temperature of the coating film on the tip of the knife 14 can be controlled to be higher than the critical temperature to change the nature of the coating film on the tip of the knife from hydrophilicity to hydrophobicity. The heater 13 can be made into a self temperature control type heater 13 by using, for example, a method of generating heat by energizing a resistive element such as a heat sensitive resistive element whose resistance increases with an increase in temperature or generating heat by supplying a high-frequency magnetic field excited by a high-frequency current to a magnetic member having a proper Curie point.

As methods of mounting a heater on a knife, a method using a printing technique and a lithography method are available. In the printing method, a resistor pattern is directly printed on the knife to form a heater. This method includes an ink-jet method and the like. In the lithography method, a resistive film is formed, and a heater pattern is formed by a removing process.

As a coating method, the same method as that in the first embodiment can be used. In this case, since it suffices if the surface of a knife is uniformly coated, this method can be executed by a relatively inexpensive method such as dipping or coating by a coater.

### Third Embodiment

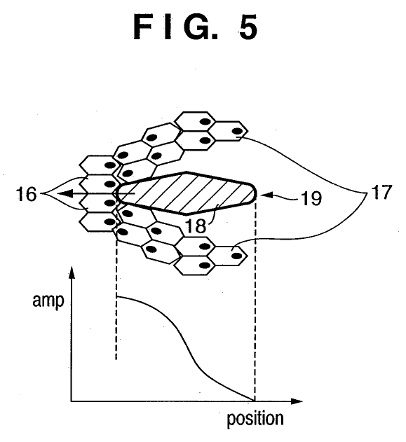

FIG. 5 is a sectional view showing how living body tissue is incised by using an ultrasonic knife according to the third embodiment. Referring to FIG. 5, reference numerals 16 and 17 denote cells; 18, a knife; and 19, a coating film made of a temperature-responsive polymer such as polyisopropylacrylamide. The graph on the lower portion of FIG. 5 represents the magnitude of the vibration amplitude in a direction perpendicular to the drawing surface at each position on the surface of the knife which corresponds to the upper portion of FIG. 5.

Incising is performed in the following manner. The maximum vibration amplitude appears at the leading end portion of the knife 18 in the traveling direction in incision, and a small vibration amplitude appears at the trailing end portion of the knife 18 in the traveling direction in incision. In this manner, longitudinal vibrations are produced in the knife 18. As a result, the temperature of the leading end portion of the knife 18 in the traveling direction in incision is raised by friction with living body tissue to exceed the above critical temperature, and hence the surface of the knife 18 becomes hydrophobic. This makes the cells 16 adhere well to the knife. On the other hand, the temperature of the trailing end portion of the knife 18 in the traveling direction in incision does not rise much (or at all) because of a small vibration amplitude. The trailing end portion therefore keeps hydrophilic, and hence the cells 17 and adhesive proteins separate well from the knife, thus preventing re-adhesion.

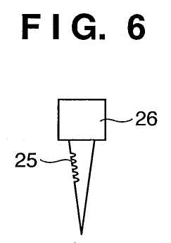

Practical methods include, for example, a method of reducing the rigidity of the leading end portion of a knife as compared with the trailing end portion by forming notches 25 or cavities in the portion (leading end portion) which comes into contact with an incision target (living body tissue), thereby increasing the vibration amplitude of the leading end portion of the knife 18, as shown in FIG. 6. FIG. 6 shows the arrangement of the knife 18, in which the knife is tapered in the longitudinal direction to enlarge vibrations from the vibration member 26 on the side in contact with the living body tissue. The first and second embodiments can also employ such a form. In this form, an excision portion used for an incision by being brought into contact with living body tissue overlaps a vibration enlarging portion which enlarges vibrations from a vibration member to produce necessary vibrations in the excision portion.

That is, as in the second embodiment, if a coating film having a critical temperature higher than the storage temperature for an excision target and lower than the temperature at which the excision target deteriorates is formed on the surface of the knife 18, and the vibration amplitude of the tip of the knife 14 is increased to generate frictional heat to make the temperature of the coating film reach a temperature higher than the critical temperature.

### Fourth Embodiment

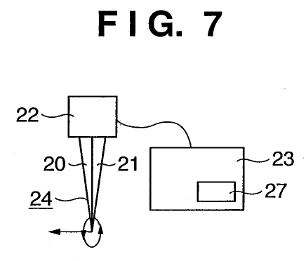

FIG. 7 is a view showing the operation of an ultrasonic knife (excision apparatus) according to the fourth embodiment. Referring to FIG. 7, reference numerals 20 and 21 denote portions of a single-piece ultrasonic knife body which have different properties. The portion 20 is covered with a hydrophobic coating. The portion 21 is covered with a hydrophilic coating. Reference numeral 22 denotes a driving device including a vibration member such as a piezoelectric element for vibrating/driving a knife 24 which is attached thereto; and 23, a driving control circuit which controls the vibrations of the vibration member and knife. The driving control circuit 23 generates elliptic vibrations in the leading end of the knife 24 as indicated by the ellipse with the arrows in FIG. 7. The driving control circuit 23 performs control to make the traveling direction (the direction indicated by the solid arrow in FIG. 7) of the knife 24 coincide with the ellipsoid of this elliptic vibration. With this control, in addition to the effect obtained by the above surface coating on the knife 24, the following effects can be obtained. As the sliding distance of the contact portion of the knife 24 which comes into contact with an excision target such as living body tissue increases, the instability of the excision direction is further reduced. In addition, dregs in an incision can be easily discharged.

Although the main body of the knife 24 has the same arrangement as that in the first embodiment, the same effects as described above can be obtained with the same arrangement as that in the second or third embodiment. In addition, if the knife according to the second embodiment is used as the knife 24, a temperature controller (or heater power supply circuit) 27 is provided in the control circuit 23 to control the temperature of the heater (or supply power to the heater).

The piezoelectric element which generates elliptic motion includes a longitudinal vibration member which vibrates in a direction vertical to the excision direction of the knife and a bending vibration member which generates bending motion parallel to the traveling direction. The arrangement of the vibration members and the shape of the knife are designed to make the resonance frequencies, that is, driving frequencies, of these vibration modes become almost equal to each other. The respective vibration members are located at antinodes of the respective vibration modes. For this reason, the excision direction almost coincides with the ellipsoid of elliptic vibration at the driving frequency. Note that vibrations can be more efficiently transmitted to the excision surface of the leading end of a knife by forming the knife into the shape of a horn such as an exponential horn which amplifies the amplitude of elliptic vibration.

As described above, according to the first to fourth embodiments, an ultrasonic knife which makes minute incision in living body tissue or the like, a vibrating knife such as an excision apparatus, and an excision apparatus have the following effects. An excision target adheres well to the incision leading end (tip) of the knife to reduce instability of the excision direction. In addition, the target after excision (incision) separates well from the knife, and hence the possibility of re-adhesion of cells and the like to the knife is low. This makes it possible to obtain a sharp cut surface, and there is little possibility of contamination.

As many apparently widely different embodiments of the present invention can be made without departing from the spirit and scope thereof, it is to be understood that the invention is not limited to the specific embodiments thereof except as defined in the appended claims.

1-2. (canceled)

3. A vibrating knife comprising:
an excision portion which is brought into contact with a target and vibrated in a direction at an angle to a traveling direction in excision so as to excise the target;
a coating which is formed on a surface of said excision portion and changes in property to hydrophobicity or hydrophilicity depending on a temperature; and
a heater which is provided on a portion of said excision portion which is located on a forward side in the traveling direction in excision, and supplies heat to said coating.

4. The knife according to claim 3, wherein said heater comprises a self temperature control type heater.

5. The knife according to claim 3, wherein said coating exhibits the change in property at a temperature higher than a storage temperature for the target and lower than a temperature at which the target deteriorates.

6-10. (canceled)

11. An excision apparatus comprising:
a vibrating knife including an excision portion which is brought into contact with a target and vibrated in a direction at an angle to a traveling direction in excision so as to excise the target, wherein a surface of said excision portion which is located on a forward side in the traveling direction in excision is hydrophobic, and a surface of said excision portion which is located on a backward side in the traveling direction in excision is hydrophilic;
a knife driving portion which vibrates said vibrating knife; and
a temperature control portion which controls generation of heat by said heater of said vibrating knife.

12-13. (canceled)

14. A method of manufacturing a vibrating knife, comprising the steps of:
forming an excision portion which is brought into contact with a target and vibrated in a direction at an angle to a traveling direction in excision so as to excise the target;
forming, on a surface of the excision portion, a film whose property changes to hydrophobicity or hydrophilicity depending on temperature; and
mounting, on a portion of the excision portion which is located on a forward side in the traveling direction in excision, a heater which supplies heat to the film.

15. (canceled)
